(19) United States
(12) Patent Application Publication
(10) Pub. No.: US 2018/0236211 A1
(43) Pub. Date: Aug. 23, 2018

(54) METHOD FOR APPLYING CONDUCTORS TO CATHETER BASED BALLOONS

(71) Applicant: Medtronic CryoCath LP, Toronto (CA)

(72) Inventor: Mark E. HENSCHEL, Phoenix, AZ (US)

(21) Appl. No.: 15/896,228

(22) Filed: Feb. 14, 2018

Related U.S. Application Data
(60) Provisional application No. 62/460,317, filed on Feb. 17, 2017.

(51) Int. Cl. A61M 25/10; A61B 18/08

(52) U.S. Cl. CPC A61M 25/1027; A61B 2018/0022; A61B 18/082

(57) ABSTRACT

The present invention advantageously provides a molding device with conductive material for creating a catheter balloon with conductive elements, and methods and systems for manufacturing the catheter balloon with conductive elements. An exemplary method for coupling a plurality of conductive elements to an expandable element may include placing a first portion of a mold proximate a second portion of the mold to define a casting cavity. Conductive material may be deposited into the casting cavity. Polymeric material may be inserted into the casting cavity. The first portion of the mold may be secured to the second portion of the mold. The polymeric material may be expanded to place the polymeric material in contact with the conductive material.

### CROSS-REFERENCE TO RELATED APPLICATION

This application is related to and claims priority to U.S. Provisional Patent Application Ser. No. 62/460,317, filed Feb. 17, 2017, entitled METHOD FOR APPLYING CONDUCTORS TO CATHETER BASED BALLOONS, the entirety of which is incorporated herein by reference.

### STATEMENT REGARDING FEDERALLY SPONSORED RESEARCH OR DEVELOPMENT

n/a

### TECHNICAL FIELD

This disclosure relates to endovascular catheters, and in particular, a method and system for applying conductive elements on catheter based balloons for tissue sensing, ablation, and cryogenic ablation.

### BACKGROUND

Developing advanced surgical tools for minimally invasive procedures may help to reduce procedure time, improve results, and reduce the likelihood of patient injury or relapse. Balloon catheters represent a frequently used class of medical instrument for delivering different therapies and facilitating various diagnoses inside the body. The catheter balloon may be inflated within a vessel and create a continuous ring of conformal contact between the tissue and the balloon. Minimally invasive insertion within the body can be achieved with a balloon catheter into internal structures. The balloon catheter may be configured through inflation to match the size and shape requirements of a particular internal structure. Sometimes the balloon is inflated and deflated, at least partially, several times during a procedure. A balloon catheter may include metal electrodes, wires, and other conductive components, which may be arranged in a variety of different ways to deliver treatments and therapies to specific tissue as well as to evaluate the tissue. The integrated circuits, sensors, actuators, and other electrical components may be coupled with a balloon catheter and in contact with tissue to provide highly localized methods of detection and therapy.

However, the stable integration of electrical components on and/or within a medical device may be difficult. For example, the size, planar geometry, and/or mechanical properties of in the electronic components and/or the medical device may complicate attachment between the electronic components and the medical device and/or cause detachment or loosening of the electronic components from the medical device during navigation through the patient's body and/or during use. Electrical components that are part of a balloon catheter may flake off or delaminate from the balloon surface during routine use and in routine conditions, which may put the patient at risk of injury. Accordingly, it would be desirable to have a way to integrate the electrical components with the medical device so they are reliably coupled and capable of performing their intended functions.

### SUMMARY

The present invention advantageously provides a molding device with electrically conductive material for creating a catheter balloon with electrically conductive elements, and methods and systems for manufacturing same. In one embodiment, a method of coupling a plurality of conductive elements to an expandable element comprises: placing a first portion of a mold proximate a second portion of the mold to define a casting cavity therebetween; depositing a conductive material into the casting cavity; inserting a polymeric material into the casting cavity; securing the first portion of the mold to the second portion of the mold; and expanding the polymeric material to place the polymeric material in contact with the conductive material.

In one aspect of the embodiment, the method further comprises stamping the conductive material into the casting cavity.

In one aspect of the embodiment, the conductive material may include a conductive material and a nonconductive material.

In one aspect of the embodiment, the conductive and the nonconductive materials are arranged in a plurality of layers to define a circuit.

In one aspect of the embodiment, the first portion and the second portion each include a semi-spherical wall defining the casting cavity.

In one aspect of the embodiment, at least a portion of the wall is coated with the electrically conductive material.

In one aspect of the embodiment, the polymeric material includes at least one of a group consisting of a thermoplastic polyurethane, a thermoplastic elastomer, a polyamide, an ethylene vinyl acetate, a polyvinylidene fluoride, and a polyvinyl chloride.

In one aspect of the embodiment, the conductive material is nitinol.

In another embodiment, a molding device comprises a mold including a first portion and a second portion defining a casting cavity therebetween, the first portion and the second portion each including an electrically conductive material disposed thereon.

In one aspect of the embodiment, the mold defines a plurality of indentations, the electrically conductive material being secured within each of the plurality of indentations.

In one aspect of the embodiment, the mold includes a securing mechanism coupling the electrically conductive material within the plurality of indentations.

In one aspect of the embodiment, the securing mechanism includes at least one of a group consisting of an adhesive, a lock, a conductive paste, a collodion, and a hardening cream.

In one aspect of the embodiment, the first portion and the second portion each include a semi-spherical wall, the semi-spherical wall of the first portion and the semi-spherical wall of the second portion together defining the casting cavity, the electrically conductive material being disposed on the semi-spherical wall of each of the first portion and the second portion.

In one aspect of the embodiment, the electrically conductive material surrounds at least a portion of the casting cavity.

In one aspect of the embodiment, the electrically conductive material is printed onto at least a portion of the first portion and the second portion of the mold.

In one aspect of the embodiment, the electrically conductive material is deposited onto at least a portion of the first portion and the second portion of the mold.

In one aspect of the embodiment, the electrically conductive material includes a conductive material and a nonconductive material.

In one aspect of the embodiment, the electrically conductive material is arranged in a plurality of layers to define a circuit.

In one aspect of the embodiment, the electrically conductive material is nitinol.

In yet another embodiment, a method of coupling a plurality of conductive elements to an expandable element comprises: printing an electrically conductive material into a pattern of an object to be cast in a casting cavity of a mold, the mold including a first portion and a second portion proximate the first portion, the first portion and the second portion together defining the casting cavity therebetween; inserting a polymeric material into the casting cavity; securing the first mold portion to the second mold portion; and expanding the polymeric material within the casting cavity thereby coupling the polymeric material with the electrically conductive material.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is an exemplary method for forming a balloon for use with a medical device with conductive elements in accordance with the principles of the present invention;

FIG. 2 is a first exemplary medical device including a balloon with conductive elements;

FIG. 3 is a second exemplary medical device including a balloon with conductive elements;

FIG. 4 is a third exemplary medical device including a balloon with conductive elements;

FIG. 5 is a fourth exemplary medical device including a balloon with conductive elements;

FIG. 6 is a fifth exemplary medical device including a balloon with conductive elements;

FIG. 7 is a sixth exemplary medical device including a balloon with conductive elements;

FIG. 8 is a flow chart showing a first exemplary method of coupling electrically conductive elements to a balloon; and

FIG. 9 is a flow chart showing a second exemplary method of coupling electrically conductive elements to a balloon.

### DETAILED DESCRIPTION

Before describing in detail exemplary embodiments, it is noted that the embodiments reside primarily in combinations of apparatus components and processing steps related to applying conductive material to a catheter balloon. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first,” “second,” “top,” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

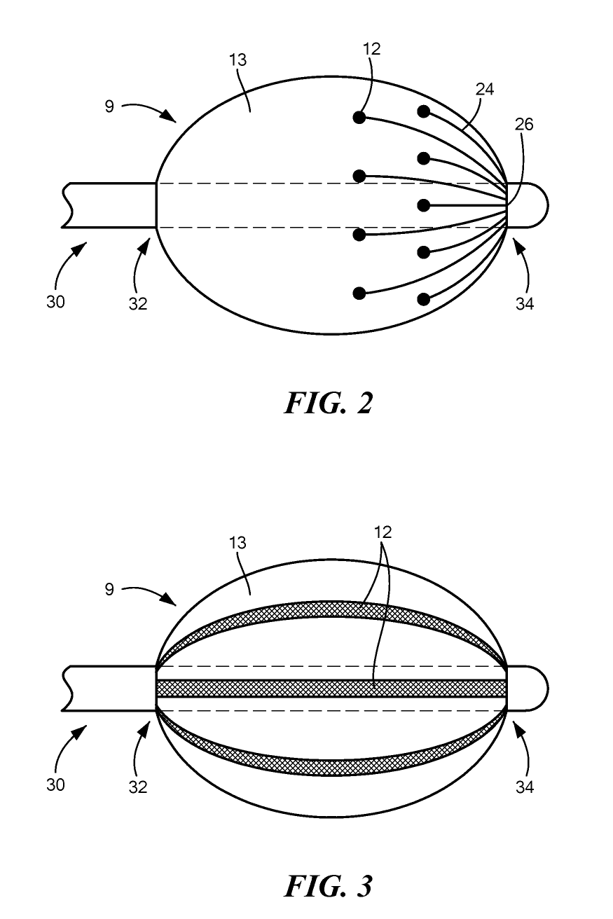

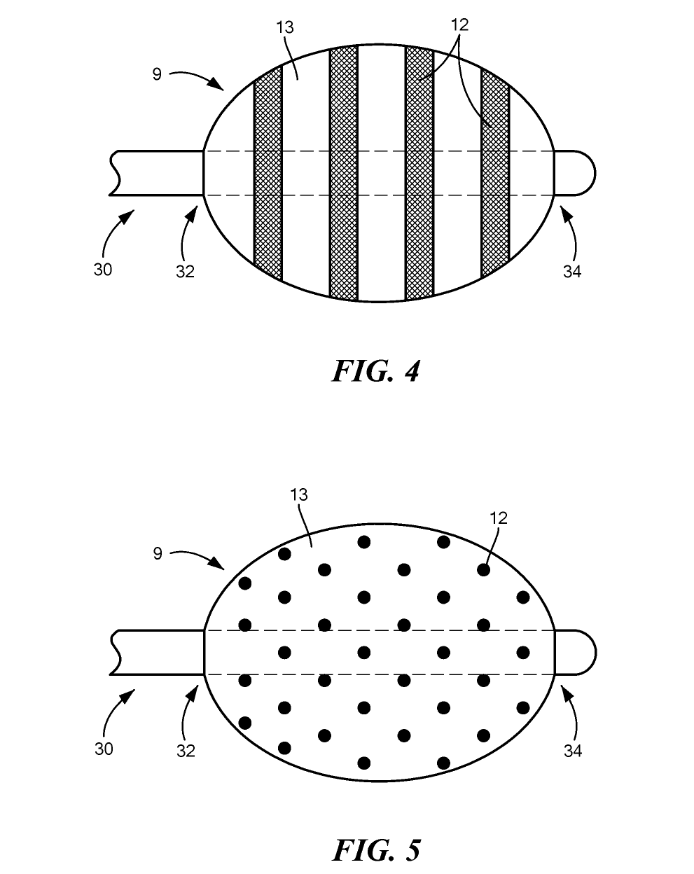

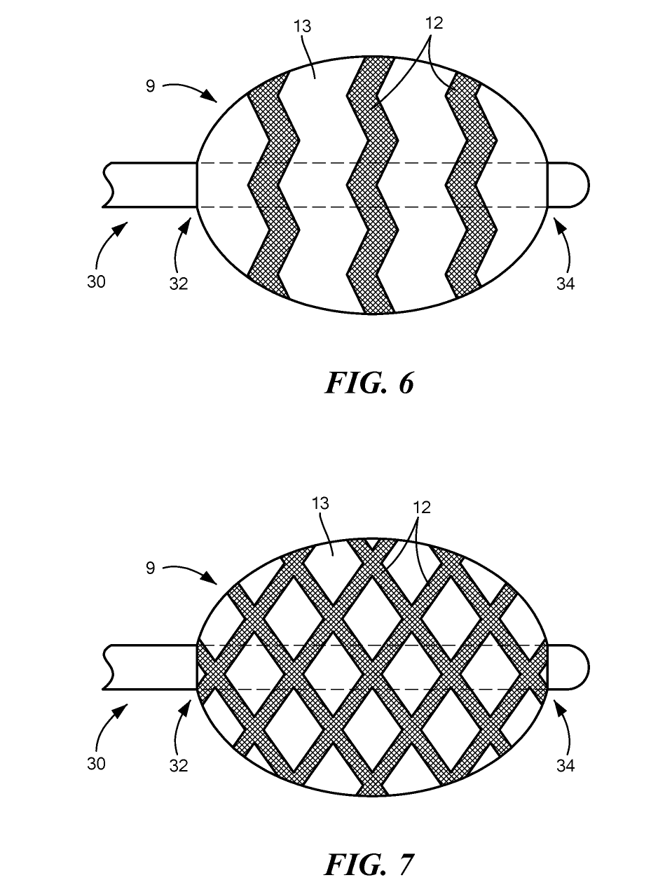

The devices, systems, and methods described herein may be used for coupling conductive elements, such as electrodes and conductive materials, to a medical device. In one embodiment, the devices, systems, and methods described herein may be used to couple conductive elements to an expandable element, such as a balloon. A balloon 9 with conductive elements may be used to, for example, treat and/or ablate tissue, record electrical signals from myocardial and other tissues, evaluate balloon-tissue contact, and/or navigate safely through the patient's vasculature. In one embodiment, a medical device includes a treatment element, such as a balloon 9, with conductive material 12, such electrodes, sensors, or other electrically and/or thermally conductive elements. Non-limiting examples of such medical device are shown in FIGS. 2-7. The balloon 9, with conductive material 12, may be used for recording data from tissue (such as mapping signals, temperature, impedance, and other signals) ablation of tissue, and cryogenic ablation of tissue and may be coupled with a control unit that is configured for use with any of a variety of energy modalities such as cryogenic, laser, or radiofrequency energy.

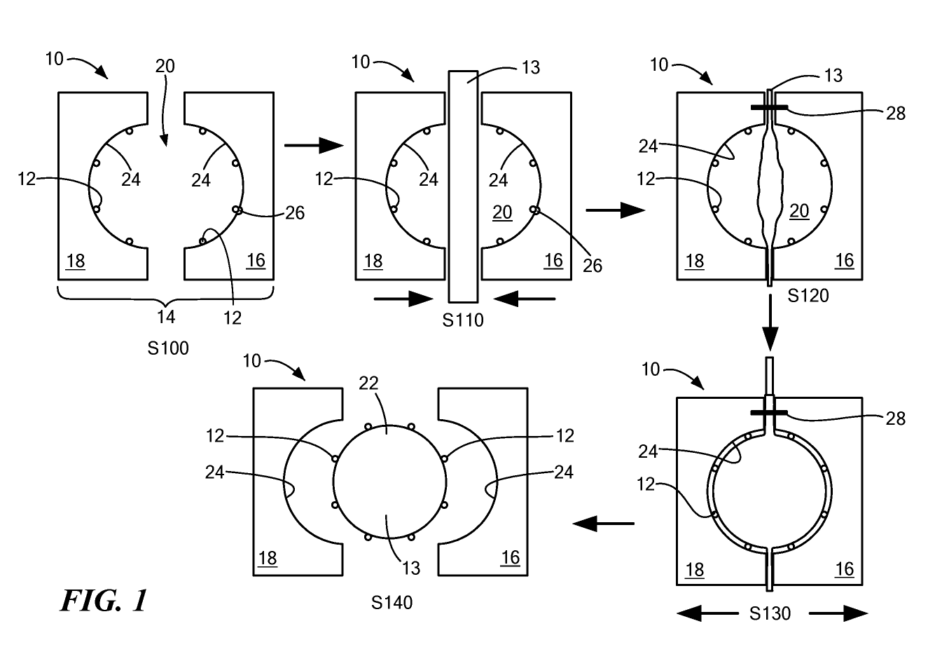

Now referring to FIG. 1, an exemplary method for forming a balloon 10 constructed in accordance with the principles of the present invention is shown, where a conductive material 12 may be in contact with polymeric material 13 as shown as S100-S140 in the figure. The polymeric material 13 with conductive material 12 (for example, electrically conductive material or thermally conductive material) may be manufactured or formed using a molding process. As shown in S100, a mold 14 may be used that has a first mold portion 16 and a second mold portion 18. The first mold portion 16 and the second mold portion 18 may be symmetrical or asymmetrical in shape and may be disposed, coupled, or secured together. The first mold portion 16 and the second mold portion 18 may have a three-dimensional shape and together create a casting cavity 20. Alternatively, the mold 14 may have any number of parts and may be made up of a single piece or more than two pieces.

The casting cavity 20 may be defined by a wall 24. The wall 24 of the casting cavity 20 may be any shape and size, including the shape of a half-circle, a half-square, a half-rectangle, and a half-oval. The casting cavity 20 may be symmetrical in shape or asymmetrical. As a non-limiting example, the casting cavity 20 may have a circular shape if it is intended that the polymeric material 13 that is used within the casting cavity 20 also will have a circular shape when inflated. Alternatively, the casting cavity 20 may have an irregular shape if the polymeric material 13 is to be inflated into an irregular shape. Thus, the determination of the size and shape of the casting cavity 20 may depend upon how and where the polymeric material 13 is going to be used. When the first mold portion 16 and the second mold portion 18 are placed proximate to one another, there may be a casting cavity 20 between them. In an exemplary embodiment, the first mold portion 16 and the second mold portion 18 may have a generally rectangular or square shape and the wall 24 of the casting cavity 20 may be different shapes and sizes, including a half-circle or a semi-circle. S100. If the first mold portion 16 and the second mold portion 18 include a semi-spherical wall which define the casting cavity 20, when the first mold portion 16 is placed proximate to the second mold portion 18 there is a casting cavity 20 between them which is the shape of a circle.

The conductive material 12 may be deposited into the wall 24 the casting cavity 20. Alternatively, the conductive material 12 may be sealed, coated, or printed onto the wall 24 of the casting cavity 20. The conductive material 12 may be deposited, coated, sealed, and/or printed onto only a portion of the wall 24 of the casting cavity 20 or on the entire wall 24 of the casting cavity 20. In one embodiment, the wall 24 includes as least one indentation 26, and the conductive material 12 may be deposited, secured, sealed, coated, printed and/or placed within the indentation(s) 26. The indentation(s) 26 may be a variety of different sizes and shapes depending upon how much conductive material 12 is to be deposited, secured, sealed, coated, printed and/or placed within the indentation 26. Generally, the amount of conductive material 12 and placement will depend upon how much is to be deposited onto the polymeric material 13. The conductive material 12 may include nitinol, gold, platinum, copper, or another biocompatible electrically conductive material 12.

As is discussed in more detail below, the conductive material 12 may be a preformed circuit, such as a circuit of stamped metal, a flexible and/or stretchable circuit, and/or any other circuit that is sized and configured to be placed into the casting cavity 20 and insert molded into a balloon 9. Additionally or alternatively, the conductive material may be inserted or deposited directly onto at least a portion of the mold 14 in the desired configuration or pattern.

The polymeric material 13 may be a natural or synthetic polymer and may include a thermoplastic polyurethane, a thermoplastic elastomer, a polyamide, an ethylene vinyl acetate, a polyvinylidene fluoride, and a polyvinyl chloride. The polymeric material 13 may be able to withstand being heated and cooled and may be able to be reformed multiple times. Additionally, if the polymeric material 13 may be able to withstand being expanded and contracted multiple times. The polymeric material 13 may serve as a thermal and electrical insulator, be light-weight, and have a significant degree of strength so that it can ultimately be used as a medical device balloon 9.

When the conductive material 12 is coated, deposited, sealed, or printed onto the wall 24 of the casting cavity 20 in the first mold portion 16 and the second mold portion 18 in different patterns and in varying amounts, the conductive material 12 may include conductive material as well as nonconductive material, even though the mixture as a whole is referred to as a conductive material 12. This conductive and nonconductive material may be arranged in various layers to define a circuit, including a flexible circuit or any other circuit. These layers of conductive and nonconductive material may insulate the conductive material 12 from one another. Also, various parts of the catheter, including an energy source such as an RF energy source, may be coupled with the conductive material 12 and may require additional insulation to allow tissue to be ablated or cryoablated with the conductive material 12. The conductive material 12 may be flexible so that it can be inserted into a portion of the casting cavity 20 as well as when it is deposited onto the polymeric material 13.

The polymeric material 13 may be inserted between the first mold portion 16 and the second mold portion 18 while they are separated from one another into the casting cavity 20. S110. When the polymeric material 13 is placed within the casting cavity 20, all of the polymeric material 13 may be inside the casting cavity 20 or a portion of the polymeric material 13 may be outside the first mold portion 16 and the second mold portion 18. The polymeric material 13 may extend above and below the first mold portion 16 and the second mold portion 18 which would allow access to a portion of the polymeric material 13 while a portion of the polymeric material 13 is inside the casting cavity 20.

As a second step, the first mold portion 16 and the second mold portion 18 may be secured to or coupled with one another with at least a portion of the polymeric material 13 within the casting cavity 20. S120. The securing mechanism 28 may include an adhesive, a lock, a conductive paste, a collodion, a hardening cream, nuts and bolts, screws and tapered holds, threaded components, wires, or any other securing mechanism.

As a third step, a solid, liquid or gas may be inserted into the polymeric material 13 to expand it to a desired shape and size. S130. A straw or other device may be inserted into the polymeric material 13 to expand it to the desired size and shape while the polymeric material 13 is inside the casting cavity 20. In one exemplary embodiment, the polymeric material 13 may be expanded into a circular or oval shape to fill the entire casting cavity 20 such that the polymeric material 13 comes into contact with the conductive material 12. When the polymeric material 13 is expanded to the desired size and shape within the first mold portion 16 and the second mold portion 18, the conductive material 12 may come into contact with polymeric material 13 and be deposited onto or otherwise adhered to the surface of the polymeric material 13 or it may be integrated within the polymeric material 13. This depositing, adhering, or integration may occur when the polymeric material 13 is expanded as the polymeric material 13 may come into contact with the conductive material 12 based upon the surface contact when the polymeric material 13 is expanded to a certain size. Alternatively, the polymeric material 13 and/or the conductive material 12 may be heated to help the conductive material 12 adhere to the polymeric material 13. Chemical treatments may also be applied to the polymeric material 13 and/or the conductive material 12 to help adhere the two to one another.

As a fourth step, the first mold portion 16 may be separated from the second mold portion 18 and the conductive material 12 that has been deposited into the polymeric material 13 may no longer be integrated into the casting cavity 20 of the first mold portion 16 and the second mold portion 18. S140. Depending upon the securing mechanism 28 used to secure the first mold portion 16 to the second mold portion 18, a releasing mechanism may be used to separate them. The pattern of the conductive material 12 on the polymeric material after it has been deposited into the polymeric material 13 may depend upon how it was deposited into wall 24 of the first mold portion 16 and the second mold portion 18.

FIGS. 2-7 provide non-limiting examples of conductive material 12 coupled with polymeric material 13 in a variety of different shapes and patterns of as part of a portion of a catheter 30. The polymeric material 13 may be a balloon 9 and may be secured to the distal end 32 of an elongated shaft 34 of a portion of a catheter 30. When the conductive material 12 is coated, deposited, or printed onto the first mold portion 16 and the second mold portion 18, a desired shape and pattern can be created. In one embodiment, the conductive material 12 may include a plurality of electrodes which may be on the surface or integrated/embedded within the polymeric material 13. Incorporating the conductive material 12 into the polymeric material 13 using the first mold portion 16 and the second mold portion 18 may prevent the conductive material 12 from flaking off the polymeric material 13. As shown as a non-limiting example in FIG. 2, the conductive material 12 may be an electrode and the electrode may be coupled with wires 36 and a multiplexer 38 to combine the signals from the different wires 36 into one signal over a shared medium.

FIGS. 2-7 show non-limiting examples of how the conductive material 12 can be coupled with the polymeric material 13 to create a balloon 9. The method as shown in FIG. 1, S100-S140 may be used to create a balloon 9 with these different patterns based upon what conductive material 12 is used and how the conductive material 12 is deposited within the casting cavity 20 of the first mold portion 16 and the second mold portion 18. A variety of different configurations of conductive material 12 on polymeric material 13 can be created using the first mold portion 16 and the second mold portion 18.

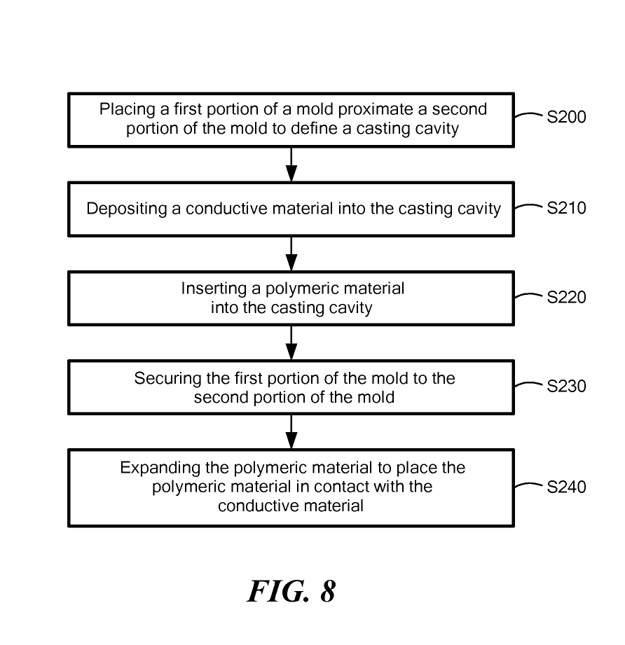

Now referring to FIG. 8, the conductive material 12 may be coupled with a polymeric material 13, including a balloon 9 using a molding method. The first mold portion 16 may be placed proximate to the second mold portion 18 to define a casting cavity 20. S200. Conductive material 12 may be deposited into the casting cavity 20. S210. The polymeric material 13 may be inserted into the casting cavity 20. S220. The first mold portion 16 may be secured to the second mold portion 18. S230. The polymeric material 13 may be expanded to place the polymeric material 13 in contact with the conductive material 12.

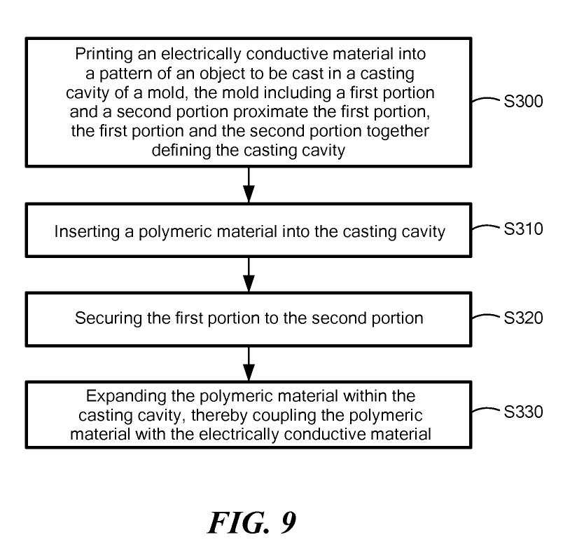

Now referring to FIG. 9, the molding may occur where conductive material 12 may be printed into a pattern of an object to be cast on a casting cavity 20 of a mold 14. The mold 14 may include a first mold portion 16 and a second mold portion 18 proximate the first mold portion 16. The first mold portion 16 and the second mold portion 18 together may define the casting cavity 20. S300. The polymeric material 13 may be inserted into the casting cavity 20. S310. The first mold portion 16 may be secured to the second mold portion 18. S320. The polymeric material 13 may be expanded within the casting cavity 20 thereby coupling the polymeric material 13 with the electrically conductive material 12. S330.

It will be appreciated by persons skilled in the art that the present embodiments are not limited to what has been particularly shown and described herein above. In addition, unless mention was made above to the contrary, it should be noted that all of the accompanying drawings are not to scale.

What is claimed is:

1. A method of coupling a plurality of conductive elements to an expandable element, the method comprising:
placing a first portion of a mold proximate a second portion of the mold to define a casting cavity therebetween;
depositing a conductive material into the casting cavity;
inserting a polymeric material into the casting cavity;
securing the first portion of the mold to the second portion of the mold; and
expanding the polymeric material to place the polymeric material in contact with the conductive material.

2. The method of claim 1, further comprising stamping the conductive material into the casting cavity.

3. The method of claim 1, wherein the conductive material includes a conductive material and a nonconductive material.

4. The method of claim 3, wherein the conductive and the nonconductive materials are arranged in a plurality of layers to define a circuit.

5. The method of claim 1, wherein the first portion and the second portion each include a semi-spherical wall defining the casting cavity.

6. The method of claim 5, wherein at least a portion of the wall is coated with the conductive material.

7. The method of claim 1, wherein the polymeric material includes at least one of a group consisting of a thermoplastic polyurethane, a thermoplastic elastomer, a polyamide, an ethylene vinyl acetate, a polyvinylidene fluoride, and a polyvinyl chloride.

8. The method of claim 1, wherein the conductive material is nitinol.

9. A molding device comprising:
a mold including a first portion and a second portion defining a casting cavity therebetween, the first portion and the second portion each including an electrically conductive material disposed thereon.

10. The device of claim 9, wherein the mold defines a plurality of indentations, the electrically conductive material being secured within each of the plurality of indentations.

11. The device of claim 10, wherein the mold includes a securing mechanism coupling the electrically conductive material within the plurality of indentations.

12. The device of claim 11, wherein the securing mechanism includes at least one of a group consisting of an adhesive, a lock, a conductive paste, a collodion, and a hardening cream.

13. The device of claim 9, wherein the first portion and the second portion each include a semi-spherical wall, the semi-spherical wall of the first portion and the semi-spherical wall of the second portion together defining the casting cavity, the electrically conductive material being disposed on the semi-spherical wall of each of the first portion and the second portion.

14. The device of claim 9, wherein the electrically conductive material surrounds at least a portion of the casting cavity.

15. The device of claim 9, wherein the electrically conductive material is printed onto at least a portion of the first portion and the second portion of the mold.

16. The device of claim 9, wherein the electrically conductive material is deposited onto at least a portion of the first portion and the second portion of the mold.

17. The device of claim 9, wherein the electrically conductive material includes a conductive material and a nonconductive material.

18. The device of claim 17, wherein the electrically conductive material is arranged in a plurality of layers to define a circuit.

19. The device of claim 9, wherein the electrically conductive material is nitinol.

20. A method of coupling a plurality of conductive elements to an expandable element, the method comprising:
printing an electrically conductive material into a pattern of an object to be cast in a casting cavity of a mold, the mold including a first portion and a second portion proximate the first portion, the first portion and the second portion together defining the casting cavity therebetween;
inserting a polymeric material into the casting cavity;
securing the first portion to the second portion; and
expanding the polymeric material within the casting cavity, thereby coupling the polymeric material with the electrically conductive material.
